(19) United States
(12) Patent Application Publication
(10) Pub. No.: US 2008/0317729 A1
(43) Pub. Date: Dec. 25, 2008

(54) Ammonium Salts and Ammonium Salt/Mineral Salt Clathrate Compounds for Use as Vehicle and Effective Form for Pharmaco-Medical Applications and for Use as Phase Transfer Agents for Chemical Applications

(76) Inventors: Helmut Kasch, Jena (DE); Uwe Reuter, Greiz (DE); Ralf Oettmeier, Greiz (DE)

Correspondence Address: JORDAN AND HAMBURG LLP, 122 EAST 42ND STREET, SUITE 4000, NEW YORK, NY, 10168, US

(21) Appl. No.: 11/658,126

(22) PCT Filed: Jul. 20, 2005
(86) PCT No.: PCT/DE2005/001288

§ 371 Date: Jan. 22, 2007

(30) Foreign Application Priority Data
Jul. 21, 2004 (DE) 10 2004 035 808.7

(51) Int. Cl. A61K 38/44; C07C 229/60; A61K 31/205; C07C 233/11; A61K 31/14; C07C 211/63; A61P 35/00

(52) U.S. Cl. 424/94.4; 560/49; 514/535; 564/194; 564/292; 514/642

(57) ABSTRACT

This invention relates to ammonium salts and stable storable ammonium salts and ammonium salt/mineral salt clathrate compounds (inclusion compounds, clusters) having acid dibasic anionic acid residues such as bicarbonate, to methods for producing them and to pharmaco-medical and chemical synthetic applications for said compounds. According to the invention, compounds for pharmaco-medical and chemical synthetic applications are produced which comprise the ammonium salt and ammonium salt/mineral salt clathrate compounds (inclusion compounds, clusters) having acid dibasic anionic acid residues of general formula I

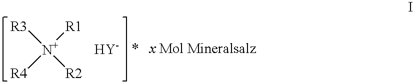

with
R1, R2, R3 and R4=alkyl and substituted alkyl straight-chain or branched, optionally having an alcohol, ether, silyether, ester, amino or amide function, H or aryl-alkyl, with aryl being an aromatic or heteroaromatic ring having optionally additional substituents, such as alkyl having 1 to 4 C atoms, OH, NR*₂ with R*₂=O, alkyl with alkyl of between 1 and 4 C atoms or H, COOH, COOR, CN, NO₂ and the cationic positive N⁺ is optionally part of an active agent, Y is a dibasic acid residue of an organic dicarboxylic acid or CO₃⁻, corresponding to HY⁻=HCO₃⁻, and x=0.5 to 30 represents the number of the mineral salt molecules for clathrate compound formation or 0. In pharmaco-medical applications applies the generalizable effectiveness principle according to which to ammonium salt/mineral salt cluster is used as vehicle and active agent for novel forms of application of nitrogen-containing active agent bases. In chemistry, these agents are used in the synthesis of active agents and valuable products, e.g. of cyclic carbonates.

The invention relates to ammonium salts and stable storable ammonium salt/mineral salt clathrate compounds (clusters, inclusion compounds) having acid dibasic acid residues such as hydrocarbonate, to methods for producing them and to pharmaco-medical and chemical synthetic applications for said compounds.

The use of ammonium salts in form of their acid salts and stable salt clusters as so called prodrugs in combination with the integrated active agent molecule is in the foreground of pharmaco-medical applications, whereas in chemical synthetic applications the attention is focused on the use of ammonium salts as phase transfer catalysts for the enantioselective or diastereoselective synthesis of active agents and valuable products, e.g. cyclic carbonates via halogen hydrins.

In pharmaco-medical applications, a lot of active agents have the disadvantage that despite of effectiveness in vitro they do not reach their real target organ in most of the administration routes because they have been metabolized during the transport to these organs and have become ineffective. To avoid said changes for an active agent, they are normally transformed into such stable products that, if it is possible, they release the active agent only in the cell or under the influence of diverse enzymes as it is the case for the liver passage. For this purpose, it is important to release the active agent in a controlled manner and to ensure a high bioavailability. Many active agents, e.g. in systemic applications, are parenterally administered and so actually transported to the target organs via the blood vessels. The active agent must be adapted to the medium surrounding it. For infusions, particularly for subcutaneous and oral administrations, the effectiveness can decrease down to the total failure of the agent. Said failure is especially often observed during the stomach passage of orally administered active agents. For basic esters of the procaine type known as local anesthetics it is known that they are cleaved particularly by esterases such as choline esterases. Up to now, prodrugs of procaine and comparable products that have led to an effect and bioavailability adequate to the ones observed in infusions, e.g. with procaine hydrochloride and sodium bicarbonate (Weber, Oettmeier, Reuter: PCT/EP 98/01742; Dhaliwal, Masih U.S. Pat. No. 5,149,320; U.S. Pat. No. 5,209,724), Shumakov, Onishchenko et. al. SU 878297; Thut, Turner U.S. Pat. No. 5,505,922), have not been published in literature. These criteria are not fulfilled by known preparations such as Novocain for which a hemolytic effect has been proven (E. R. Hammerland and K. Pederson-Bjergard; J. pharm. Sci 1961, 50, 24), not by the alkaline and moderately dissoluble diprocainium carbonate, also known under the name Jenacain, and not by procaine active agent conjugates (Kasch, Goldschmidtt: PCT/EP 00/13036) either. The use of so called modifiers (PCT/US 93/05631) has not led to a successful result so far. Procaine and its analog products have various biological effects that cannot be made use of sufficiently if they are parenterally administered because said pharmacological products are badly resorbed and therefore they have a poor bioavailability. Among other reasons, this disadvantage is due to their restricted solubility and their tendency to be precipitated. In addition to this, freshly prepared infusion solutions, e.g. in combination with bases, are only stable for a limited time and are metabolized even during 30 minutes at temperatures>30° C., with transformation into p-aminobenzoic acid and diethyl aminoethanol. To prevent the decomposition of analog procaine products, particularly procaine hydrochloride, stabilizers such as benzyl alcohol are added but they can lead to unintended side effects, e.g. allergies. In many cases, an unbalanced and disturbed isotony and/or isohydry are/is another cause.

In chemical synthetic applications, e.g. in the production of cyclic carbonates from halogen hydrins that are required e.g. for PET (positron-emission tomography), work-intensive, dangerous and low effective methods (transformation with phosgen, urethanu production, rearrangement reactions) are used. Enantioselective syntheses or diastereoselective syntheses have not been performed till now. Phase-transfer salts, by means of which cyclic carbonates are produced from vicinal halogen hydrins, are not of technical significance due to their low yields. Thus, only poor or no transformations could be observed if tetrabutylammonium halogen (halogen=Cl⁻, Br⁻ or I) and NaHCO₃ have been used.

The task of this invention is to describe compounds and methods for producing them as well as pharmaco-medical and chemical synthetic applications that allow the better utilization of the potential of active nitrogenous bases, such as procaine, lidocaine or diethyl aminoethanol, and thus help to overcome the disadvantages known from the state of the art.

According to this invention, the object of this invention is achieved by compounds for pharmaco-medical and chemical synthetic applications, comprising ammonium salts and ammonium salt/mineral salt clathrate compounds (clusters, inclusion compounds) having acid dibasic anionic acid residues of general formula I.

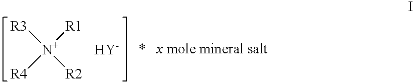

with R1, R2, R3 and R4=alkyl and substituted alkyl straight-chain or branched, optionally having additional alcohol, ether, silyether, ester, amino or amide function, H or aryl-alkyl with aryl, with aryl being an aromatic or heteroaromatic ring having optionally additional substituents, such as alkyl having 1 to 4 C atoms, OH, NR*₂ with R*₂=O-alkyl with alkyl of between 1 and 4 C atoms or H, COOH, COOR, CN, NO₂ and the cationic positive N⁺ is optionally part of an active agent.

Y=a dibasic acid residual of an organic dicarboxylic acid or CO₃⁻, corresponding to HY⁻=HCO₃⁻,

and x=0.5 to 30 represents the number of the mineral salt molecules for clathrate compound formation or 0.

Apart from/and/or instead of the mineral salts that are useful for clathrate compound formation, special embodiments of the compounds of this invention can contain dextrans, cellulose ester or starch, e.g. cornstarch, as stabilizing substances tending to the formation of clathrate compounds.

The compounds of this invention can contain monovalent, bivalent and trivalent metal salt cations such as Na⁺, K⁺, Li⁺, Mg⁺⁺, Ca⁺⁺, Zn⁺⁺, Fe⁺⁺, Fe⁺⁺⁺, Mn⁺⁺ and as anions Cl⁻, Br⁻, J⁻, F⁻, SO₄⁻, SO₃⁻, HSO₃⁻, HCO₃⁻, PO₄³⁻, HPO₄⁻, H₂PO₄⁻, SiO₄⁴⁻, AlO₂⁻, SiO₃ and/or [(AlO₂)₁₂(SiO₂)₂]²⁻ as mineral salts that are useful for the formation of clathrate compounds.

Ammonium salts and ammonium salt/mineral salt clusters are used as the inventive compounds for pharmaco-medical purposes and said compounds are derived from bases as active components, with procaine, substituted procaines, epinephrine, tetracaine, lidocaine, bupivacain, pontocaine, propoxycaine, octacaine, mepivacaine, prilocaine, dibucaine, isocaine, marcaine, etidocaine, piridocaine, eucaine, butacaine, cocaine, articaine, N,N-diethyl aminoethanol, N,N-dimethyl aminoethanol, N-ethyl, N-methyl aminoethanol or N,N-diethyl aminopropargyl with free and protected alcohol function that can be esterified, etherified or silylated, being considered as active agent bases, and ammonium salts and ammonium salt/mineral salt clusters containing tetraalkyl ammonium hydrogen carbonates are used for chemical synthetic applications.

Preferred compounds for pharmaco-medical and chemical synthetic applications of the above mentioned inventive compounds are:
procainium—[ProcH]⁺ (fluorine, chlorine, bromine or iodine procainium-) bicarbonate
N-alkyl-procainium [Alkyl-Proc]⁺ bicarbonate
lidocainium [LidocainH]⁺ bicarbonate,
N-alkyl lidocainium-[alkyl lidocain]⁺ bicarbonate.

Another object of this invention is to provide a method to produce the inventive compounds. According to this invention, in said method mineral acid ammonium salts, such as NR₄HSO₄, NR₄HSO_{3,} (NR₄)₂HPO₄, NR₄H₂PO₄, NR₄halogen with halogen=Cl, Br, I, and/or organo-acid ammonium salts, such as NR₄Tosylat, NR₄OCO-(Alkyl)-COOH, with R₄ in the above indicated meaning for R1, R2, R3 and R4 and with alkyl=0 through 12 C atoms with NaHCO₃, NH₄HCO₃, Ca(HCO₃)_{2,} Mg(HCO₃)₂ or KHCO₃ in a suitable solvent, optionally by the addition of CO₂ also in form of dry ice under pressure and for the stabilization of required salts, are transformed into the corresponding mono-, di-, tri-substituted or quaternary ammonium bicarbonates.

The inventive tetraalkyl ammonium bicarbonates, such as tetrabutyl ammonium bicarbonates or N-alkyl procainium bicarbonates, are preferably produced by the transformation of NR₄HSO₄ with NaHCO₃ or NH₄HCO₃, with tetraalkyl ammonium bicarbonates being preferred for chemical synthetic applications in situ in an aprotonic solvent, e.g. acetonitrile, and said inventive tetraalkyl ammonium bicarbonates are directly used as reagents for substrates, such as racemic and optically active trans-1,2- or trans-1,3-halogenhydrin, with halogen=Cl, Br or I, for the stereospecific production of cyclic carbonates.

The inventive ammonium bicarbonate/mineral salt clathrate compounds (inclusion compounds, clusters) of the above mentioned general formula I, NR₄HCO₃ x mineral salt, are primarily brought to transformation in the cold temperature range by transforming mineral acid or dibasic organo-acid ammonium salts in the presence of metal⁺ and/or metal⁺⁺-bicarbonates, preferably alkali or alkaline earth bicarbonates, and/or ammonium bicarbonate plus the addition of carbon dioxide (H₂O/CO₂), which is produced under pressure, and possibly further mineral salts and/or dextrans, cellulose esters or starch, and afterwards said inventive compounds are dehydrated by water bonding preparations, e.g. mineral salts, or by freeze drying. Due to the stabilizing effect of the mineral salts in form of clathrate compounds (inclusion compounds, clusters), also dextrans, cellulose esters or starch, said compounds are obtained as stable, storable solids for pharmaco-medical and chemical synthetic applications.

According to this invention, mono-, di-, tri-alkyl ammonium bicarbonates can be produced in situ in the cold temperature range by transforming the basic amines, e.g. procaine, lidocaine or diethylaminoethanol, with ammonium bicarbonate (NH₄HCO₃) and/or carbonic acid, also by adding dry ice/water, but for the transformation into solid, stable and storable salts a mineral salt or dextran, cellulose ester or starch, e.g. cornstarch, must be added to them before dehydration as a stabilizing medium tending to clathrate compounds.

According to this invention, mineral salt clathrate compounds (clusters) containing procainium-, lidocainium- or N,N-diethyl,N-(1-hydroxyethyl)ammonium-bicarbonate can be produced by the transformation of procaine, lidocaine or diethylaminoethanol with ammonium bicarbonate and stabilizing mineral salts in the cold temperature range and solid stable salt clusters are obtained after dehydration.

The inventive compounds can be used for pharmaco-medical applications for fighting against pains and inflammatory processes, against acidosis, tumor diseases, cardiovascular diseases, autoimmune diseases due to a reduced host defense, for convalescence and “wellness” purposes, for stress prophylaxis and as an “antiaging” means in geriatrics.

The compounds that can be produced according to the invention for pharmaco-medical applications can be used both in a solid state for oral, dermal, nasal, anal or lingual administrations or in a dissolved state, also as suspensions, for parenteral and peritoneal administrations or for inhalations. For these purposes, further carrier substances, stabilizers, diluting agents and other auxiliary means that are usual for the field of medicaments, can be contained and, if it is possible, the compounds should be prepared to the exclusion of protic diluting agents and extreme heating and moisture should be avoided, short applications such as infusions, injections or inhalations excluded.

The compounds producible according to the invention for pharmaco-medical applications can use endogenic substances of the respiratory chain, such as CO₂ and HCO₃, as well as excess bicarbonate in the salt cluster for the transport of the active agent the bioavailability of which is even improved by the additional administration of carbonic anhydrase inhibitors.

The solid compounds, salt clusters, that can be produced according to the inventive method and are also suited for the preparation of infusion and injection solutions, for tablets or powders and implants, represent active clusters that contribute to a better targeting and an improved bioavailability thanks to the controlled release of the active agent.

The solid compounds that can be produced according to the invention for pharmaco-medical applications are used in active agent doses of between 0.01 mg and 2000 mg and have an improved tolerability and therapeutical breadth.

In the following, some explanations are given about the substantial steps by means of which the mentioned problems of the state of the art have been surprisingly overcome.

It was surprisingly found that substituted amines, primary, secondary, tertiary and quaternary amines that can also function as a component of biologically active agents, also acid salts of them, can be transformed into ammonium salts (NR₄HCO₃) with bicarbonate being the anion and in this form or in the form of stable clusters they represent a new quality. Due to their specific properties they can be used for new medical and chemical applications. These salts (salt clusters) can be produced according to the following total formulas:

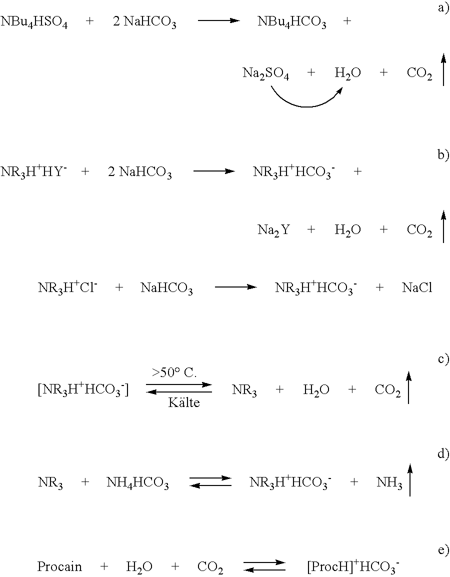

The her formulated transformations in an anhydrous or hydrous medium would not be remarkable if it was not known from the general state of the art that ammonium bicarbonates and also correspondingly N-substituted compounds are considered to be very unstable and till now there has been doubt about their production in solid form. Now, it has been found that N-substituted ammonium bicarbonates develop according to the above transformation formulas. This fact can be proven by analytic physicochemical data gained for example by the determination of the conductivity and the freezing point depression, in mass spectrometric measurements, UV and IR measurements as well as NMR measurements in D₂O that have been used for structure clarification purposes. But the above formulas also show—and this can be proven by the identification of the corresponding substances—that the production of the solid stable and dry substances in a quality that is required for pharmaco-medical and chemical applications is not possible without problems because they can again decompose into their components. Mainly, this decomposition is observed if the specific reaction conditions for obtaining the target substances are not meticulously kept!

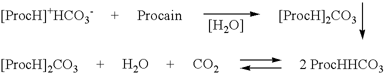

If the proportion of procaine cannot be kept low or to zero and/or the water cannot be kept away from the procainium hydrocarbonate simultaneously, diprocainium carbonate, which precipitates in hydrous solutions, is formed easily. Due to the increased basicity of diprocainium carbonate the saponification of procaine is also initiated. To avoid this process during the production of the ammonium salt clusters or to invert it, carbonic acid or CO₂, also in the form of dry ice, and water are added.

An inventive solution is the integration of the actually instable compounds such as procainium or lidocainium hydrocarbonate into mineral salts and/or dextrans, starch or cellulose to so called clathrate compounds or clusters. Surprisingly, this object is successfully achieved even with simple mineral salts such as sodium chloride. In these salts, clusters, the ammonium salts are either integrated into the salt lattice in a coordinative manner or they are covered and enclosed. In comparison to the infusion solutions used up to now and prepared by mixing hydrous solutions of procaine hydrochloride and sodium carbonate at room temperature, the proportion of strongly basic substances such as procaine and carbonates, e.g. diprocainium carbonate, could be reduced significantly thanks to physicochemical modifications. Even small quantities of carbonate catalyze the decomposition of procainium bicarbonate into procaine or diprocainium carbonate. The production procedure, the decrease of the pH by the addition of CO₂, forms the condition to increase the content of NR₄HCO₃ up to more or less 100% and by the integration into salts or other compounds that are able to form clusters it is preserved in a sophisticated, original manner adequate to biological systems.

The ammonium bicarbonates existing in the salt clusters latently contain carbonic acid that cannot be released under normal conditions at room temperature, but in aqueous solution and even more in organic-aqueous solutions they are released quickly.

On the one hand, the active agent can be released in a controlled manner from the clathrate compounds and be used for pharmaco-medical applications, and on the other hand the CO₂ or the bicarbonate can be used for chemical synthetic purposes, e.g. as a reagent for the stereoselective production of cyclic carbonates.

Kinetic investigations about the metabolism of the active agent clusters demonstrate that the stability of the dissolved compounds is sufficient under physiological conditions to select the systemic or also local administration of the active agent, e.g. procaine, and to ensure an optimum transport to the targets. If the active agent fixed in the clusters, including carbonic acid, is dissolved in water it forms a typical acid-base pair that exhibits a high buffer capacity even if a surplus of bicarbonate exists. The acid-base pair can be used to maintain or to correct the physiological pH, e.g. in case of acidosis, and moreover it has excellent properties like its good solubility that is a basic condition for a good bioavailability. The well-balanced physiological system of the venous blood, the CO₂/HCO₃ balance included, is not affected by the salt clusters. The system is in fact useful for the stabilization of the acid-base pair.

Choline esterases that can be purposefully controlled by esterase inhibitors, thus also by carboanhydrase inhibitors, are influenced by the excess of bicarbonate. This influence causes a lowered enzyme effectiveness and increases indirectly the separation and consequently also the lifetime or availability of e.g. procainium salt.

The existence of stable and water-soluble N-substituted ammonium bicarbonates of the procainium bicarbonate salt cluster type in combination with additional bicarbonate in form of for example sodium bicarbonate allows a patient-friendlier use of the active agents and does not stay limited to injections and infusions. Due to a defined, exact analytically provable composition of the solutions that can be produced from salt clusters, an improvement of the state of the art known up to now has been reached here, too. The physiological tolerability and the harmlessness of the salt clusters used in the indicated dose range are demonstrated in pH measurements and toxicological investigations. Depending on the additionally used bicarbonate, the pH value of the infusion solution can be set and kept constant by the acid-base pair of the salt cluster within a pH range of between 7.3 and 8.3. Apart from the already mentioned applications, this possibility allows the careful therapeutic use for acidosis.

Among other reasons, the failing of hemolisis in comparison to procaine hydrochloride is caused by the low toxicity due to the high buffer capacity of the salt cluster with procainium bicarbonate used as the active component. Investigations made with a dark-field microscope show that the erythrocytes remain intact even in case of a high salt excess and do not burst.

Toxicological studies concerning a chicken embryo show on the one hand the heart effectiveness that, however, becomes apparent by an increase of the heart frequency for a short time and decreases quickly, not least for the dilatory effect of the procainium bicarbonate salt cluster, and on the other hand they show the tolerability. Thus, defect blood vessels, which have been injured e.g. by the addition of an oil suspension of the salt clusters, have been repaired under the influence of the active agent.

In application investigations, the efficacy of the salt clusters has been checked after ensuring that the tolerability is even better guaranteed than for the use of procaine hydrochloride. By using the salt clusters in form of a powder, as capsules or tablets this threshold is still further decreased. It is not absolutely necessary to cover the tablets to avoid a possible decomposition when the gastrointestinal tract is passed, because during the pressing process a protection layer is formed that is preferably decomposed in the intestine.

For nasal applications the powder that can be administered as a nose spray is useful on the one hand, on the other hand the inhalation of the powder dissolved in sodium chloride (active agent content of 65 mg procaine/inhalation) or an appropriate tablet is a suitable method for a local (marginally also systemic) application in the nose and nasal sinus areas. In this way, pains and inflammations can be treated in the nasal sinus and the spreading of pains to adjacent areas (headache, toothache) can be avoided. Up to now, procaine/base injections have been used for this type of application, but the use of the salt cluster solution as an alternative is a patient-friendlier and optimum method.

Corticoids with all their side effects are used for the systemic treatment of inflammations, such as arthritis, multiple sclerosis (MS), chronic inflammatory diseases of the intestine, inflammations of the nerve tracts or inflammations of the spinal cord. In long-term systemic or local application, procaine clusters can cause a comparable anti-inflammatory effect even here. The unpleasant side effects of the corticoids do not appear in this method.

The prophylactic administration of procaine clusters reduces the consequences of the spreading or establishment of diseases that are caused by stress, e.g. tinnitus. Among other reasons, the effect of the proc clusters is due to the neurogenic and antioxidative effect of the active agent bases. An excess of sodium bicarbonate stimulates this process, a fact that is proven by investigations of macrophage (chemiluminescence at PMNL cells).

The stabilization of N-substituted ammonium bicarbonates by means of cluster formation does not only allow the production of solid forms of these compounds that have been considered instable up to now, but due to the varied properties it also opens a significantly better bioavailability, e.g. as a physiologically adapted carrier and transport form. Aqueous solutions can be prepared from the clusters or clathrate compounds for injections and infusions without using adverse additives. Another advantage of said compounds is their use as a reagent for the stereoselective synthesis of 1.3- and 1.2-cis (Z) cyclic carbonates for PET (positron-emission tomography).

Now, the invention is explained in more detail by means of the following embodiments that do not restrict the invention in any way:

### I For Chemical Synthetic Applications of Tetraalkyl Ammonium Bicarbonates

### EXAMPLE 1

### 16β,17β-carbonyldioxy-3-methoxy-estra-1,3,5(10)-trien

11 g 16α-bromine,17β-hydroxy-3-methoxy-estra-1,3,5(10)-trien (30.11 mmol) are dissolved in 50 ml acetonitrile and stirred after the addition of 10 g Bu₄NHSO₄ and 20 g NaHCO₃ at room temperature for 16 hours. The Bu₄NHCO₃ produced in situ reacts diastereoselectively to cis (Z)-cyclic carbonate with the also produced Na₂SO₄ being responsible for binding trace amounts of water. After the successful transformation, the suspension is filtered, the residue is washed with acetonitrile and the filtrate is mixed into ca. 100 ml finely crushed ice. To achieve the complete crystallization, the product is left in the refrigerator for about 16 hours and 8 g of 16β,17β-cyclic carbonate, which can be recrystallized from methanol/methylene chloride, are obtained.

Mp: 145 to 150° C.

IR [cm⁻¹]: 1496, 1604 (aromatic), 1788 (cycl. carbonate)

MS [m/z]: ES⁻ 341.5 (M−H; calculated for M=342.48)

### EXAMPLE 2

### 16β,17β-carbonyldioxy-3-methoxymethyloxy-estra-1,3,5(10)-trien

3 g 16α-bromine,17β-hydroxy-3-methoxy-estra-1,3,5(10)-trien (7.6 mmol) are dissolved in 50 ml acetonitrile and stirred after the addition of 3 g Bu₄NHSO₄ and 6 g NaHCO₃ at room temperature for 16 hours. After the successful transformation, the suspension is filtered, the residue is washed with acetonitrile and the filtrate is mixed into ca. 50 ml finely crushed ice. To achieve the complete crystallization, the product is left in the refrigerator for about 16 hours and 2 g of 16β,17β-cyclic carbonate, which can be recrystallized from ethyl acetate, are obtained. The cyclic carbonate is used for the production of precursors for PET (positron-emission tomography).

Mp: 111 to 115° C.

IR [cm⁻¹]: 1496, 1604 (aromatic), 1790 (cycl. carbonate)

MS [m/z]: ES⁻ 357.5 (M−H; calculated for M=358.44)

### EXAMPLE 3

### 16α,17α-carbonyldioxy-3-methoxymethyloxy-estra-1,3,5(10)-trien

3 g 16α-bromine,17β-hydroxy-3-methoxymethyloxy-estra-1,3,5(10)-trien (7.6 mmol) are brought to transformation by analogy with example 2. After precipitation of the oily residue, the 16α,17α-cyclic carbonate is filtered in a frit and recrystallized from ethyl acetate.

IR [cm⁻¹]: 1496, 1604 (aromatic), 1789 (cycl. carbonate)

MS [m/z]: ES⁻ 357.5 (M−H; calculated for M=358.44)

### EXAMPLE 4

### 16β,17β-carbonyldioxy-3-methoxymethyloxy-5-androsten

3 g 16α-bromine,17β-hydroxy-3-methoxymethyloxy-5-androsten (7.25 mmol) are dissolved in 50 ml acetonitrile and stirred after the addition of 3 g N-ethyl procainium bicarbonate [also producible in situ from N-ethyl procainium bisulphate and NaHCO₃ or N-ethyl procainium iodide, NaHSO₄, NaHCO₃) at room temperature for 30 hours. After the successful transformation, the suspension is filtered, the residue is washed with acetonitrile and the filtrates are combined. To isolate the steroid, ether and water are added for extraction purposes and after separation and evaporation of the organic solvent the remaining residue is chromatographed on silica gel. A toluene/ethyl acetate mixture (30:1) is used as the elution means. The obtained result are 900 mg of the 16β,17β-cyclic carbonate crystallizing from ethyl acetate/hexane.

Mp: 144 to 147° C.

IR [cm⁻¹]: 1789 (cycl. carbonate)

MS [m/z]: ES⁻ 375.6 (M−H; calculated for M=376.5)

### II For Pharmaco-Medical Applications

### EXAMPLE 5

### Procainium Bicarbonate Salt Cluster

### a) Proc*HCl (NaHCO₃H₂O Under Pressure)

100 ml of a cooled aqueous solution saturated with CO₂ under pressure are added to 5.456 g procaine hydrochloride (20 mmol) at a temperature of between 0° C. and −4° C. and afterwards 6.721 g NaHCO₃ are added. Then, the clear homogeneous solution is frozen and freeze-dried. The freeze drying is performed until a constant weight is obtained, i.e. a decrease of the weight cannot be observed any longer. 11.9 g (97.7% of th.) of the salt cluster containing procainium bicarbonate are obtained and can be directly used for pharmaco-medical and chemical synthetic applications. For pharmaco-medical applications, the salt cluster is suited for tablets on the one hand, with the tablet coating itself with a covering during the pressure process and thus allowing a passage through the stomach. On the other hand, the salt cluster is also suited for the preparation of injections and infusions. In these cases it is possible to select a hypotonic administration by the addition of water and an isotonic administration by the addition of sodium bicarbonate or isotonic salt solution.

Thermoanalysis (of 0.609 g= 1/20 of the preparation): 22.2 ml CO₂=0.99 mmol accordingly 0.99 mmol procainium bicarbonate for 1/20 of the preparation quantity are released!

¹H-NMR (D₂O) [ppm]: 7.89, 7.86 (d); 6.86, 6.83 (d); 4.59 (tr); 3.48 (tr); 3.21 (qu); 1.295 (tr)

¹³C-NMR (D₂O) [ppm]: 9.093 (2*CH₃), 48.83 (2*CH₂₎), 50.817 (1*CH₂), 60.14 (1*CH₂), 115.143 (2*aromat. CH), 132.258 (2*arom. CH), 160.781; 153.336 (2*quat. arom. C), 168.655 (OC═O)

MS [m/z]: ES⁺: 237.7 (236+H); 259.7 (236+Na)

### b) Procaine/Carbonic Acid (NaHCO₃)

236 mg procaine (1 mmol) are suspended in 30 ml water and cooled down to 0° C. during its introduction into the solution until the procaine is completely dissolved. The end of the reaction, i.e. the formation of procainium bicarbonate, is determined by conductivity measurements and the definition of the freezing point depression (increased conductivity due to salt formation, noticeable freezing point depression). As during the freeze drying of the procainium bicarbonate/carbonic acid solution the procainium bicarbonate decomposes into its components procaine, CO₂ and water, a homogeneous solution containing 4 mmol NaHCO₃ is added in the cold temperature range. The clear solution is frozen and afterwards freeze-dried with the excess CO₂ being removed in vacuum. As a result, 0.65 g (95.6% of Th) of the salt cluster containing procainium bicarbonate are obtained.

### c) Procaine/H₂SO₄/NaHCO₃/CO₂

236 mg procaine (1 mmol) are suspended in 5 ml water and 2 ml of 1 n H₂SO₄ are added by cooling it down to 0° C. At a temperature of between 0° C. and −4° C., 5 ml of a cooled homogeneous solution saturated with CO₂ under pressure and containing 0.336 g NaHCO₃ (4 mmol) are added to the clear solution. The clear solution is frozen and afterwards freeze-dried until a decrease of the weight cannot be observed any longer. As a result, 0.57 g (94.2% of Th) of the salt cluster containing procainium bicarbonate are obtained.

### d) Procaine/NaHSO₄/NaHCO₃/CO₂

7 ml of an aqueous solution containing 120.05 mg NaHSO₄ are added to 236 mg procaine (1 mmol). At a temperature of between 0° C. and −4° C., 5 ml of a cooled homogeneous solution saturated with CO₂ under pressure and containing 0.336 g NaHCO₃ (4 mmol) are added to the clear solution. The clear solution is frozen and afterwards freeze-dried until a decrease of the weight cannot be observed any longer. As a result, 0.54 g (91% of Th) of the salt cluster containing procainium bicarbonate are obtained.

### e) Procaine/CO₂/Water/NaHCO₃/NaCl

4.72 g procaine (20 mmol) are suspended to about 5° C. in 100 ml water and simultaneously cooled, afterwards 5.04 g NaHCO₃ (60 mmol) and 1.168 g NaCl are added and also cooled. In intervals of 10 minutes, dry ice in portions of 0.25 cm³ is given to the suspension by stirring it strongly. The procedure is repeated till all the procaine has dissolved. The clear solution is frozen and afterwards freeze-dried. The freeze drying is performed until-a constant weight is obtained, i.e. a decrease of the weight cannot be observed any longer. As a result, 12 g (98.3% of Th) of the salt cluster containing procainium bicarbonate are obtained and can be directly used for pharmaco-medical and chemical synthetic applications.

f) 49.102 g procaine hydrochloride are dissolved in 2000 ml aqueous carbonic acid saturated with CO₂ and in the cold temperature range treated with 60.49 g NaHCO₃ and 172.6 g NaCl. Afterwards, the clear solution is frozen and afterwards freeze-dried. The freeze drying is performed until a constant weight is obtained, i.e. a decrease of the weight cannot be observed any longer. As a result, 281.24 g (99.63% of Th) of the salt cluster containing procainium bicarbonate are obtained and can be directly used for pharmaco-medical applications, particularly for the preparation of an isotonic infusion solution, (1.15 g proc cluster in 100 ml water).

### EXAMPLE 6

### Lidocainium Bicarbonate Salt Cluster

2.705 g lidocaine hydrochloride (10 mmol) are dissolved in ca. 5 ml water and at a temperature of between 0° C. and −4° C., a cooled homogeneous solution saturated with CO₂ under pressure and containing 3.361 g NaHCO₃ (40 mmol) are added. Afterwards, the clear solution is gradually frozen and excess CO₂ is removed in vacuum. The reaction mixture is freeze-dried until a decrease of the weight cannot be observed any longer. As a result, 5.65 g (93% of Th) of the salt cluster containing lidocainium bicarbonate are obtained.

Thermoanalysis (of 0.607g= 1/10 of the preparation): 21.2 ml CO₂=0.95 mmol accordingly 0.95 mmol lidocainium bicarbonate for 1/10 of the preparation quantity.

MS [m/z]: E⁺ 236 (M+H); 258 (M+Na)

### EXAMPLE 7

### N,N-Diethyl,N-(1-hydroxyethyl)ammonium Bicarbonate Salt Cluster

a) 1.76 g (15.04 mmol) N,N-diethyl,N-(1-hydroxyethyl)-amin(diethyl aminoethanol) are neutralized with an equivalent quantity of diluted hydrochloric acid and at a temperature of between 0° C. and 4° C., 100 ml of a carbonic acid solution saturated with CO₂ are added. Afterwards, 6.721 g (80 mmol) NaHCO₃ are added and the preparation is stirred until the bicarbonate is completely dissolved. Then, the clear solution is frozen and freeze-dried. The freeze drying is performed until a constant weight is obtained, i.e. a decrease of the weight cannot be observed any longer. As a result, 8.7 g (96.34% of Th) of the salt cluster containing N,N-diethyl,N-(1-hydroxyethyl)ammonium bicarbonate are obtained and can be directly used for pharmaco-medical and chemical synthetic applications. In pharmaco-medical applications, the salt cluster is suitable for tablets. The tablets are to be stored under cool conditions to prevent their decomposition.

CO₂ release: 1.85 ml CO₂ are released from 0.0501 g salt cluster, This corresponds to a content of 100% salt cluster

b) 100 ml of a carbonic acid solution saturated with CO₂ are added to 1.76 g (15.04 mmol) N,N-diethyl,N-(1-hydroxyethyl)-amine(diethyl aminoethanol) at a temperature of between 0° C. and 4° C. Afterwards, dry ice in portions of about 0.25 cm³ is given to the suspension in intervals of 10 minutes. This procedure is repeated approximately eight times and then 5.055 g NaHCO₃ (60 mmol) and 0.879 g NaCl (15.04 mmol) are added and the reaction mixture is stirred at about 5° C. until the salts are completely dissolved. Afterwards, the clear solution is frozen and freeze-dried. The freeze drying is performed until a constant weight is obtained, i.e. a decrease of the weight cannot be observed any longer. As a result, 8.3 g (96.39% of Th) of the salt cluster containing N,N-diethyl,N-(1-hydroxyethyl)-ammonium bicarbonate are obtained and can be directly used for pharmaco-medical applications. In pharmaco-medical applications, the salt cluster is suitable for tablets. The tablets are to be stored under cool conditions to prevent their decomposition.

1. Compound for pharmaco-medical and chemical synthetic applications, comprising an ammonium salt or ammonium salt/mineral salt clathrate compound having an acid dibasic anionic acid residue of the general formula I, 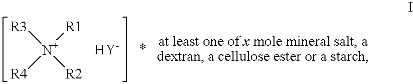
wherein
R1, R2, R3 and R4=alkyl or substituted alkyl straight-chain or branched, optionally having additional alcohol, ether, silyether, ester, amino or amide function, H or aryl-alkyl, with aryl being an aromatic or heteroaromatic ring having optionally at least one additional substituent, the additional substituent being alkyl having 1 to 4 C atoms, OH, NR*₂ with R*₂=O-alkyl of 1 to 4 C atoms or H, COOH, COOR, CN, or NO₂ and the cationic positive N⁺ is optionally part of an active agent.
Y=a dibasic acid residual of an organic dicarboxylic acid or CO₃⁻, corresponding to HY⁻=HCO₃⁻,
x=0.5 to 30 represents the number of the mineral salt molecules for clathrate compound formation and
the ammonium salt or ammonium salt/mineral salt clathrate compound is derived from a base as active component, the active component comprising at least one of procaine, substituted procaines, epinephrine, tetracaine, lidocaine, bupivacaine, pontocaine, propoxycaine, octacaine, mepivacaine, prilocaine, dibucaine, isocaine, marcain, etidocaine, piridocaine, eucaine, butacaine, cocaine, articaine, N,N-diethyl aminoethanol, N,N-dimethyl aminoethanol, N-ethyl, N-methyl aminoethanol or N,N-diethyl aminopropargyl with free and protected alcohol function that can be esterified, etherified or silylated.

2. (canceled)

3. Compound as set forth in claim 1, wherein the mineral salt comprises at least one monovalent, bivalent or trivalent cation selected from the group consisting of Na⁺, K⁺, Li⁺, Mg⁺⁺, Ca⁺⁺, Zn⁺⁺, Fe⁺⁺, Fe⁺⁺⁺, Mn⁺⁺ and as anions Cl⁻, Br⁻, I⁻, F⁻, SO₄⁻, SO₃⁻, HSO₃⁻, HCO₃⁻, PO₄³⁻, HPO₄⁻, H₂PO₄⁻, SiO₄⁴⁻, AlO₂⁻, SiO₃⁻ and [(AlO₂)₁₂(SiO₂)₂]²⁻.

4. Method for producing a compound as set forth in claim 1, wherein ammonium salt/mineral salt comprising at least one mineral acid ammonium salts selected from the group consisting of NR₄HSO₄, NR₄HSO_{3,} (NR₄)₂HPO₄, NR₄H₂PO₄, and NR₄halogen, wherein halogen=Cl, Br, I, and/or ammonium salt comprising at least one organo-acid ammonium salt selected from the group consisting of NR₄Tosylat and NR₄OCO-(Alkyl)-COOH, wherein R4 is alkyl of up to 12 C atoms, with NaHCO₃, NH₄HCO₃, Ca(HCO₃)_{2,} Mg(HCO₃)₂ or KHCO₃ in a solvent, optionally by adding CO₂ as gas or dry ice under pressure and for the stabilization of required salts, is transformed into the corresponding mono-, di-, tri-substituted or quaternary ammonium bicarbonate.

5. Method for producing a compound as set forth in claim 1, wherein a tetraalkyl ammonium bicarbonate is produced by transformation of NR₄HSO₄ with NaHCO₃ or NH₄HCO₃, or a tetraalkyl ammonium bicarbonate for chemical synthetic applications in situ in an aprotonic solvent and used as a reagent for a substrate selected from the group consisting of racemic and optically active trans-1,2- or trans-1,3-halogenhydrin, wherein halogen=Cl, Br or I, for the stereospecific production of a cyclic carbonate.

6. Method for producing a compound as set forth in claim 4 or 5, wherein an ammonium bicarbonate/mineral salt clathrate of the formula I, NR₄HCO₃ x mineral salt, is primarily brought to transformation in a cold temperature range by transforming mineral acid or dibasic organo-acid ammonium salts in the presence of metal⁺ and/or metal⁺⁺-bicarbonates plus the addition of H₂O/CO₂, which is prepared under pressure, and optionally further at least one of mineral salts and/or dextrans, cellulose esters or starch, and afterwards dehydrated by a water binding agent compound or by freeze drying, and due to the stabilizing effect of the mineral salts in form of clathrate compounds, also a dextran, cellulose ester or starch, said compound is obtained as a stable, storable solid for pharmaco-medical and chemical synthetic applications.

7. Method for producing a compound as set forth in claim 4 or 5, wherein a mono-, di- and tri-alkylammonium bicarbonate is produced in a cold temperature range in situ by transformation of a basic amine with ammonium bicarbonate and/or carbonic acid, plus the addition of dry ice/water, and for the transformation into a solid, stable and storable salt a mineral salt or dextran, cellulose ester or starch is added as a stabilizing substance for formation of a clathrate compound before a dehydrating process.

8. A pharmaco-medical product comprising a compound of claim 1 wherein the dibasic anionic residue comprises hydrocarbonate.

9. A method of treating pain, inflammation, acidosis, tumor diseases, cardiovascular diseases, autoimmune diseases due to a reduced host repulse, or for convalescence and “wellness” purposes, for stress prophylaxis or “antiaging” in geriatrics, comprising administering a pharmaco-medical product comprising a compound of claim 1.

10. A method of claim 9, wherein the pharmaco-medical product is in a solid state and the administration is oral, dermal, nasal, anal or lingual, or the pharmaco-medical product is solution or suspension and the administration is parenteral or peritoneal or by inhalation.

11. A method of claim 9, wherein the compound comprises at least one endogenic substance of the respiratory chain as well as excess bicarbonate for transport of active agent.

12. A method of claim 9, wherein the pharmaco-medical product comprises an infusion or injection solution, tablet or powder or implant providing controlled release of active agent with reduced doses of the active agent.

13. A method of claim 9, wherein the pharmaco-medical product comprises 0.01 mg to 2000 mg of active agent, wherein improved tolerability and bioavailability are provided.

14. A method of claim 11, further comprising administering a carbonic anhydrase thereby to increase bioavailability of the active agent.
